(19) United States
(12) Patent Application Publication
(10) Pub. No.: US 2022/0331365 A1
(43) Pub. Date: Oct. 20, 2022

(54) BIOMIMETIC NANOEMULSIONS FOR OXYGEN DELIVERY

(71) Applicant: The Regents of the University of California, Oakland, CA (US)

(72) Inventors: Liangfang Zhang, La Jolla, CA (US); Ronnie H. Fang, La Jolla, CA (US); Jia Zhuang, La Jolla, CA (US); Weiwei Gao, La Jolla, CA (US)

(21) Appl. No.: 17/639,895

(22) PCT Filed: Sep. 1, 2020
(86) PCT No.: PCT/US2020/048906

§ 371 Date: Mar. 2, 2022

Related U.S. Application Data
(60) Provisional application No. 62/895,094, filed on Sep. 3, 2019.

(51) Int. Cl. A61K 35/18; A61K 9/127; A61K 31/02; A61P 9/10

(52) U.S. Cl. CPC A61K 35/18; A61K 9/127; A61K 31/02; A61P 9/10

(57) ABSTRACT

A biomimetic oxygen delivery carrier is provided by employing natural cell membrane as a stabilizer for fluorocarbon nanoemulsions. The resulting formulation exhibits a high capacity for delivering oxygen and can be used to successfully resuscitate subjects in need due to for example hemorrhagic shock. This natural-synthetic platform can alleviate the impact of blood shortages in clinical settings among other uses.

### CROSS REFERENCE TO RELATED APPLICATIONS

This application claims the priority benefit of U.S. Provisional Application No. 62/895,094, filed Sep. 3, 2019, which application is incorporated herein by reference.

### GOVERNMENT SPONSORSHIP

This invention was made with government support under grant CA200574 awarded by the National Institutes of Health. The government has certain rights in the invention.

### TECHNICAL FIELD

The present invention relates to nanoemulsions including fluorocarbon as an oxygen delivery vehicle enveloped in a stabilizing cellular membrane.

### BACKGROUND

Ever since methods for the typing and storage blood were developed early in the 20^{th} century, blood transfusions have become an essential part of modern medicine.^{[1]} The ability of donated red blood cells (RBCs) to restore oxygen transport capacity is a life-saving measure for patients who have lost significant blood volume. The procedure is commonly employed in cases of acute trauma and during surgical procedures.^{[2, 3]} Currently, donated RBCs, particularly those from universal donor types, are a precious resource that have a limited shelf-life under standard storage conditions, and cancellations of elective surgeries are common in times of short supply.^{[4]} Although efforts to lessen blood utilization by doctors and supply chain management improvements can help to reduce shortages,^{[5, 6]} these methods alone are not expected to fully address the issue. As such, significant efforts have also been placed on finding alternative strategies to help reduce the demand for timely human donations.^{[7-10]} Promising candidates have included platforms based on hemoglobin and perfluorocarbons (PFCs), although both have been met with significant challenges in terms of clinical translation. Currently, Fluosol-DA represents the only synthetic oxygen carrier to have been approved by the United States Food and Drug Administration; however, it was taken off the market 5 years after approval in 1989 due to difficulties in its storage and use.^{[11]}

PFC emulsions are attractive for oxygen delivery applications due to their inertness, inherent ability to solubilize gases, and small size.^{[12-15]} As a result of their chemical structure, PFCs are highly hydrophobic and lowly reactive, giving them the capability to dissolve large amounts of gases such as oxygen and carbon dioxide. Compared with water, many PFCs have nearly 20 times the capacity for oxygen dissolution. As this is a physical process, a larger proportion of the carried oxygen is generally available for release to the tissues when compared with hemoglobin, which follows a sigmoidal dissociation curve.^{[16]} Further, PFC emulsions can be fabricated at the nanoscale,^{[17, 18]} and this small size enables them to deliver oxygen even to the smallest of capillaries. Despite their advantages, PFC-based platforms generally have not experienced much clinical success, which can largely be attributed to issues such as difficulty of storage and adverse immune reactions.^{[9]}

### SUMMARY OF THE INVENTION

The invention provides a hybrid natural-synthetic nanodelivery platform that combines the biocompatibility of natural RBC membrane with the oxygen carrying ability of fluorocarbons. The resulting formulation can be stored long-term and exhibits a high capacity for oxygen delivery, helping to mitigate the effects of hypoxia in vitro. In an animal model of hemorrhagic shock, mice are resuscitated at an efficacy comparable to whole blood infusion. By leveraging the advantageous properties of its constituent parts, this biomimetic oxygen delivery system can address a critical need in the clinic.

In embodiments, the invention provides novel nanoparticles, and methods of using and making novel nanoparticles. More specifically, the inventive nanoparticle comprises a) an inner core comprising a non-cellular oxygen delivery vehicle; and b) an outer surface comprising a cellular membrane or hybrid membrane derived from a cell. In certain embodiments, the inner core of the inventive nanoparticle comprises a biocompatible and/or a synthetic oxygen delivery vehicle including, but not limited to, a fluorocarbon, such as a perfluorocarbon (PFC), e.g., perfluorooctyl bromide, and any other suitable derivative thereof, or synthetic material or the like. Alternative fluorocarbons can be used as an oxygen delivery vehicle, including, but not limited to, perfluorooctyl bromide (C8F17Br, also referred to as perflubron), perfluorodecyl bromide (C10F21Br) and perfluorodichlorooctane (C8F16C12).

In certain embodiments, the outer surface of the inventive nanoparticle comprises a cellular membrane comprising a plasma membrane or an intracellular membrane derived from a unicellular (e.g., a bacterium or fungus) or multicellular organism (e.g., a plant, an animal, a non-human mammal, a vertebrate, or a human). In certain embodiments, the outer surface of the inventive nanoparticle comprises a naturally occurring cellular or viral membrane and/or further comprises a synthetic membrane. In certain embodiments, the outer surface comprises a hybrid membrane. A hybrid membrane is a membrane in which the membrane shell comprises two or more different types of cellular membranes or comprises one or more naturally occurring cellular membrane and a synthetic lipid membrane. In certain embodiments, the cell membrane is an engineered cell membrane, where genetic engineering is used to modify the cells and then collect the membrane.

In certain embodiments, the cellular membrane of the outer surface of the inventive nanoparticle is derived from a blood cell (e.g., red blood cell (RBC), white blood cell (WBC), or platelet). In other embodiments, the cellular membrane of the outer surface is derived from an immune cell (e.g., macrophage, monocyte, B-cell, or T-cell), a tumor or cancer cell, and other cells, such as an epithelial cell, an endothelial cell, or a neural cell. In other embodiments, the cellular membrane of the outer surface is derived from a non-terminally differentiated cell, such as a stem cell, including a hematopoietic stem cell, a bone marrow stem cell, a mesenchymal stem cell, a cardiac stem cell, or a neural stem cell. The non-terminally differentiated cell can be isolated in a pluripotent state from tissue or induced to become pluripotent. In yet other embodiments, the cellular membrane is derived from a cell component or cell organelle including, but not limited to, an exosome, a secretory vesicle, a synaptic vesicle, an endoplasmic reticulum (ER), a Golgi apparatus, a mitochondrion, a vacuole or a nucleus.

In certain embodiments, the present invention further provides that the inventive nanoparticle comprises a releasable cargo that can be located in any place inside or on the surface of the nanoparticle. A trigger for releasing the releasable cargo from the inventive nanoparticle includes, but is not limited to, contact between the nanoparticle and a target cell, tissue, organ or subject, or a change of an environmental parameter, such as the pH, ionic condition, temperature, pressure, and other physical or chemical changes, surrounding the nanoparticle. In certain embodiments, the releasable cargo comprises one or more of a therapeutic agent, prophylactic agent, diagnostic or marker agent, prognostic agent, e.g., an imaging marker, or a combination thereof. In yet certain other embodiments, the releasable cargo is a metallic particle, a polymeric particle, a dendrimer particle, or an inorganic particle.

The present nanoparticle can have any suitable shape. For example, the present nanoparticle and/or its inner core can have a shape of sphere, square, rectangle, triangle, circular disc, cube-like shape, cube, rectangular parallelepiped (cuboid), cone, cylinder, prism, pyramid, right-angled circular cylinder and other regular or irregular shape. The present nanoparticle can have any suitable size.

The present invention further provides that in certain embodiments the inventive nanoparticle has a diameter from about 10 nm to about 10 μm. In certain embodiments, the diameter of the invention nanoparticle is about 50 nm to about 500 nm. In other embodiments, the diameter of the nanoparticle can be about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, and 10 μm, or any suitable sub-ranges within the about 10 nm to about 10 μm range, e.g., a diameter from about 50 nm to about 150 nm. In certain embodiments, the inner core supports the outer surface.

The present invention further provides that the inventive nanoparticle substantially lacks constituents of the cell from which the cellular membrane is derived or constituents of the virus from which the viral membrane is derived. For example, the present nanoparticle can lack, in terms of types and/or quantities, at least 10%, 20%, 30%, 40%, 50%, 60%, 70%, 80%, 90%, 95%, 96%, 97%, 98%, 99% or 100% of the constituents of the cell from which the cellular membrane is derived or constituents of the virus from which the viral membrane is derived.

In yet certain other embodiments, the nanoparticle of the present invention substantially maintains natural structural integrity or activity of the cellular membrane, the membrane derived from a virus or the constituents of the cellular membrane or viral membrane. The structural integrity of the cellular membrane includes primary, secondary, tertiary or quaternary structure of the cellular membrane, the membrane derived from a virus or the constituents of the cellular membrane or viral membrane, and the activity of the cellular membrane includes, but is not limited to, binding activity, receptor activity, signaling pathway activity, and any other activities a normal naturally occurring cellular membrane, the membrane derived from a virus or the constituents of the cellular membrane or viral membrane, would have. In certain embodiments, the nanoparticle of the present invention is biocompatible and/or biodegradable. For example, the present nanoparticle can maintain, in terms of types and/or quantities, at least 10%, 20%, 30%, 40%, 50%, 60%, 70%, 80%, 90%, 95%, 96%, 97%, 98%, 99% or 100% of the natural structural integrity or activity of the cellular membrane, the membrane derived from a virus or the constituents of the cellular membrane or viral membrane.

In certain embodiments, the nanoparticle of the present invention comprises the cellular plasma membrane derived from a red blood cell and an inner core comprising a fluorocarbon, such as a perfluorocarbon (PFC), e.g., perfluorooctyl bromide perfluorodecyl bromide, or perfluorodichlorooctane, wherein the nanoparticle substantially lacks hemoglobin. For example, the present nanoparticle can lack, in terms of types and/or quantities, at least 10%, 20%, 30%, 40%, 50%, 60%, 70%, 80%, 90%, 95%, 96%, 97%, 98%, 99% or 100% of the hemoglobin of the red blood cell from which the plasma membrane is derived.

In certain embodiments, the invention nanoparticle substantially lacks immunogenicity to a species or subject from which the cellular membrane is derived. For example, the present nanoparticle can lack, in terms of types and/or quantities, at least 10%, 20%, 30%, 40%, 50%, 60%, 70%, 80%, 90%, 95%, 96%, 97%, 98%, 99% or 100% of the immunogenicity to a species or subject from which the cellular membrane is derived.

The present invention further provides a medicament delivery system, and/or a pharmaceutical composition comprising the inventive nanoparticle. In certain embodiments, the medicament delivery system and/or the pharmaceutical composition of the present invention further comprises one or more additional active ingredients and/or a medically or pharmaceutically acceptable carrier or excipient, which can be administered along with or in combination with the nanoparticle of the present invention.

The present invention further provides a method for treating and/or preventing a disease or condition in a subject in need using the inventive nanoparticles, the medicament delivery system, or the pharmaceutical composition comprising the same. In certain embodiments, the cellular membrane of the nanoparticle used for the inventive method is derived from a cell of the same species of the subject or is derived from a cell of the subject. In certain embodiments, the cellular membrane of the nanoparticle used for the inventive method is derived from a red blood cell of the same species of the subject and the red blood cell has the same blood type of the subject. In certain embodiments, the nanoparticle, the medicament delivery system, or the pharmaceutical composition is administered via any suitable administration route. For example, the nanoparticle, the medicament delivery system, or the pharmaceutical composition can be administered via an oral, nasal, inhalational, parental, intravenous, intraperitoneal, subcutaneous, intramuscular, intradermal, topical, or rectal route. In certain embodiments, the disease or condition is decompression sickness, sickle cell crisis, surgery, trauma, cancer oxygen sensitizer, and/or other hypoxia related conditions.

In certain embodiments, the invention nanoparticle substantially lacks immunogenicity to a species or subject from which the cellular membrane is derived. For example, the present nanoparticle can lack, in terms of types and/or quantities, at least 10%, 20%, 30%, 40%, 50%, 60%, 70%, 80%, 90%, 95%, 96%, 97%, 98%, 99% or 100% of the immunogenicity to a species or subject from which the cellular membrane is derived.

The present invention further provides a medicament delivery system, and/or a pharmaceutical composition comprising the inventive nanoparticle. In certain embodiments, the medicament delivery system and/or the pharmaceutical composition of the present invention further comprises one or more additional active ingredients and/or a medically or pharmaceutically acceptable carrier or excipient, that can be administered along with or in combination with the nanoparticle of the present invention.

The present invention further provides a method for treating and/or preventing a disease or condition in a subject in need using the inventive nanoparticles, the medicament delivery system, or the pharmaceutical composition comprising the same. In certain embodiments, the cellular membrane of the nanoparticle used for the inventive method is derived from a cell of the same species of the subject or is derived from a cell of the subject. In certain embodiments, the cellular membrane of the nanoparticle used for the inventive method is derived from a red blood cell of the same species of the subject and the red blood cell has the same blood type of the subject. In certain embodiments, the nanoparticle, the medicament delivery system, or the pharmaceutical composition is administered via any suitable administration route. For example, the nanoparticle, the medicament delivery system, or the pharmaceutical composition can be administered via an oral, nasal, inhalational, parental, intravenous, intraperitoneal, subcutaneous, intramuscular, intradermal, topical, or rectal route.

In other embodiments, the nanoparticle is administered via a medicament delivery system. In yet other embodiments, the inventive method further comprises administering another active ingredient, or a pharmaceutically acceptable carrier or excipient, to the subject in need. The inventive method further provides that the nanoparticle of the present invention can be administered systemically or to a target site of the subject in need. Use of an effective amount of nanoparticles of the present invention for the manufacture of a medicament for treating or preventing a disease or condition in a subject in need is also provided.

Furthermore, the present invention provides an immunogenic composition comprising an effective amount of nanoparticle that comprises an inner core comprising a non-cellular material, and an outer surface comprising a cellular or plasma membrane derived from a cell and an antigen or a hapten. A vaccine comprising the immunogenic composition of the present invention is also provided. The present invention further provides a method of use of the invention immunogenic composition for eliciting an immune response to the antigen or hapten in a subject in need of such elicitation, and method of use of the invention vaccine comprising the immunogenic composition for protecting a subject against the antigen or hapten. In certain embodiments, the immune response is T-cell or B-cell mediated immune response. Use of an effective amount of the nanoparticle of the present invention for the manufacture of the immunogenic composition against an antigen or hapten, and use of an effective amount of the immunogenic composition for the manufacture of a vaccine for protecting a subject against the antigen or hapten, are also provided.

The present invention further provides a method for making the nanoparticle of the invention, comprising mixing a nanoparticle inner core comprising a non-cellular material with a cellular membrane derived from a cell or a membrane derived from a virus while exerting exogenous energy to form the nanoparticle. In certain embodiments, the exogenous energy is a mechanical energy, e.g., a mechanical energy exerted by extrusion. In other embodiments, the exogenous energy is an acoustical energy, e.g., an acoustical energy exerted by sonication. In yet other embodiment, the exogenous energy is a thermal energy, e.g., a thermal energy exerted by heating. In yet other embodiments, the inventive method further comprises mixing a nanoparticle inner core comprising non-cellular material with a naturally occurring cellular membrane derived from a cell or a naturally occurring membrane derived from a virus with a synthetic membrane while exerting exogenous energy to form the nanoparticle comprising the inner core and an outer surface comprising the cellular membrane or viral membrane and the synthetic membrane.

The present invention further provides a neoplasm specific immunogenic composition comprising an effective amount of the nanoparticle that comprises an inner core comprising a non-cellular material, and an outer surface comprising a cellular membrane derived from a neoplasm cell, wherein the cellular membrane substantially retains its structural integrity for eliciting an immune response to the neoplasm cell. For example, the present nanoparticle can maintain, in terms of types and/or quantities, at least 10%, 20%, 30%, 40%, 50%, 60%, 70%, 80%, 90%, 95%, 96%, 97%, 98%, 99% or 100% of its structural integrity for eliciting an immune response to the neoplasm cell.

In certain embodiments, the inner core supports the outer surface of such nanoparticles. In certain embodiments, the inner core of such nanoparticles comprises PFC and the outer surface comprises a plasma membrane derived from a neoplasm cell. In other embodiments, the outer surface of such nanoparticles comprises naturally occurring cellular or viral membrane and further comprises a synthetic membrane.

In certain embodiments, the inner core supports the outer surface, and the cellular membrane in the outer surface of the nanoparticle substantially retains its structural integrity for substantially retaining the toxin. In yet certain other embodiments, the outer surface of the nanoparticle comprises a naturally occurring cellular or viral membrane and further comprises a synthetic membrane or synthetic or naturally occurring components added to the cellular membrane. In yet certain other embodiments, the nanoparticle contained in such pharmaceutical composition is biocompatible, biodegradable, or comprises a synthetic material. In yet certain other embodiments, the pharmaceutical composition of the present invention further comprises another active ingredient or a pharmaceutically acceptable carrier or excipient.

The present invention contemplates treatments, prevention, diagnosis and/or prognosis of any diseases, disorders, or physiological or pathological conditions, including, but not limited to, blood loss, hemorrhagic shock, trauma, an infectious disease, a parasitic disease, a neoplasm, a disease of the blood and blood-forming organs, a disorder involving the immune mechanism, endocrine, nutritional and metabolic diseases, a mental and behavioral disorder, a disease of the nervous system, a disease of the eye and adnexam, a disease of the ear and mastoid process, a disease of the circulatory system, a disease of the respiratory system, a disease of the digestive system, a disease of the skin and subcutaneous tissue, a disease of the musculoskeletal system and connective tissue, a disease of the genitourinary system, pregnancy, childbirth and the puerperium, a condition originating in the perinatal period, a congenital malformation, a deformation, a chromosomal abnormality, an injury, a poisoning, a consequence of external causes, and an external cause of morbidity and mortality.

In some embodiments, the present nanoparticles, medicament delivery systems, pharmaceutical compositions and methods, can be used to deliver the exemplary medications listed in the Orange Book: Approved Drug Products with Therapeutic Equivalence Evaluations (Current through March 2012) published by the U.S. Food and Drug Administration, the exemplary medications listed in The Merck Index (a U.S. publication, the printed 14th Edition, Whitehouse Station, N.J., USA) and its online version (The Merck Index Online℠, Last Loaded on Web: Tuesday, May 1, 2012), and the exemplary medications listed in Biologics Products & Establishments published by the U.S. Food and Drug Administration, and can be used to treat or prevent the corresponding diseases and disorders.

### BRIEF DESCRIPTION OF THE DRAWINGS

FIGS. 1a-1e. Formulation of RBC-PFC. FIG. 1a shows a schematic illustration of oxygen delivery and release to hypoxic tissues by RBC-PFC. FIG. 1b shows a diameter of RBC-PFC at various PFC to RBC membrane ratios (n=3, mean+s.d.). FIG. 1c shows a diameter of RBC-PFC after various emulsification times (n=3, mean+s.d.). FIG. 1d show images of RBC membrane vesicles, bare PFC emulsions mixed with RBC vesicles, and RBC-PFC after centrifugation at 600 g; the RBC membrane was labeled with DiD. FIG. 1e shows confocal fluorescence imaging of dual-labelled RBC-PFC; the RBC membrane was labelled with DiD, and the PFC core was labelled with BODIPY (grayscale)). Scale bar, 1 μm.

FIGS. 2a-2f. RBC-PFC characterization. FIG. 2a shows a diameter of RBC vesicles, bare PFC emulsions, and RBC-PFC (n=3, mean+s.d.). FIG. 2b shows Zeta potential of RBC vesicles, bare PFC emulsions, and RBC-PFC (n=3, mean+s.d.). FIG. 2c shows quantification of perfluorooctyl bromide (left) loading by ¹⁹F-NMR, where perfluoro-15-crown-5-ether (right) was used as an internal standard; the fluorine atoms corresponding to each respective peak are colored in blue (grayscale). FIG. 2d shows stability of bare PFC emulsions and RBC-PFC over the course of 96 days (n=3, mean±s.d.). FIG. 2e shows dissolved oxygen kinetics after the addition of oxygenated water, RBC vesicles, PFC emulsions, RBC-PFC, or whole RBCs into deoxygenated water. FIG. 2f shows dissolved oxygen kinetics after the addition of RBC-PFC fabricated from in-dated or outdated human RBCs, as well as human RBC-PFC after storage for 1 week at either room temperature (RT) or 4° C.

FIGS. 3a-3h. In vitro oxygen delivery using RBC-PFC. FIG. 3a shows viability of Neuro2a cells after incubation with RBC-PFC at various concentrations for 24 h (n=6; mean±s.d.). FIG. 3b shows cytokine levels produced by J774 macrophages after incubation with PFC emulsions or RBC-PFC for 24 h (n=3; mean+s.d.). ****p<0.0001; one-way ANOVA. FIG. 3c shows viability of Neuro2a cells after different hypoxia induction periods, followed by incubation with RBC-PFC at various concentrations for 24 h under hypoxic conditions (n=6; mean±s.d.). *p<0.05, ****p<0.0001 (6 h vs. 18 h); ##p<0.01, ###p<0.001, ####p<0.0001 (6 h vs. 24 h); &p<0.05, &&p<0.01, &&&&p<0.0001 (18 h vs. 24 h); one-way ANOVA. FIG. 3d shows a western blot for HIF1α expression in Neuro2a cells subject to hypoxia, hypoxia in the presence of RBC-PFC following an 18 h induction period, or normoxia. MW, molecular weight in kDa. FIGS. 3e and 3g show Brightfield microscopy of Neuro2a cells before and 24 h after being subject to hypoxia, hypoxia in the presence of RBC-PFC, or normoxia; cells were subject to either 0 h (FIG. 3e) or 18 h (FIG. 3g) of hypoxia induction. Scale bars, 200 μm. FIGS. 3f and 3h show fluorescence microscopy of Neuro2a cells before and 6 h after being subject to hypoxia, hypoxia in the presence of RBC-PFC, or normoxia; cells were labeled with Image-iT Green hypoxia reagent (greyscale) and were subject to either 0 h (FIG. 3f) or 18 h (FIG. 3h) of hypoxia induction. Scale bars, 200 μm.

FIGS. 4a-4g. In vivo oxygen delivery and safety of RBC-PFC. FIG. 4a shows mean arterial pressure (MAP) profiles of mice after blood withdrawal, followed by infusion with Ringer's lactate (RL), RBC vesicles, PFC emulsions, RBC-PFC, or whole blood (n=6; mean±s.d.). FIG. 4b shows MAP values for each group at the endpoint of (a) (n=6; mean+s.d.). *p<0.05, ****p<0.0001; one-way ANOVA. FIG. 4c shows biodistribution of DiD-labeled RBC-PFC in major organs, including the liver, spleen, heart, lungs, kidneys, and blood, at various times after administration (n=6; mean+s.d.). FIG. 4d shows serum cytokine levels over time after intravenous administration of isotonic sucrose (vehicle), PFC emulsions, or RBC-PFC (n=3; mean±s.d.). ***p<0.001; one-way ANOVA. FIG. 4e shows comprehensive blood chemistry panel taken 24 h after intravenous administration of isotonic sucrose or RBC-PFC (n=3; mean+s.d.). ALB: albumin, ALP: alkaline phosphatase, ALT: alanine transaminase, AMY: amylase, TBIL: total bilirubin, BUN: blood urea nitrogen, CA: calcium, PHOS: phosphorus, CRE: creatinine, GLU: glucose, NA⁺: sodium, K⁺: potassium, TP: total protein, GLOB: globulin (calculated). FIG. 4f shows counts of various blood cells 24 h after intravenous administration of isotonic sucrose or RBC-PFC (n=3; geometric mean+s.d.). WBC: white blood cells, RBC: red blood cells, PLT: platelets. FIG. 4g shows hematoxylin and eosin (H&E) staining of histology sections from major organs 24 h after RBC-PFC administration. Scale bar, 250 μm.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a pharmaceutical composition, and/or a method that “comprises” a list of elements (e.g., components, features, or steps) is not necessarily limited to only those elements (or components or steps), but may include other elements (or components or steps) not expressly listed or inherent to the pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein the term “pharmaceutical composition” refers to a pharmaceutical acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “subject” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

Cellular Membrane: The term “cellular membrane” as used herein refers to a biological membrane enclosing or separating structure acting as a selective barrier, within or around a cell or an emergent viral particle. The cellular membrane is selectively permeable to ions and organic molecules and controls the movement of substances in and out of cells. The cellular membrane comprises a phospholipid uni- or bilayer, and optionally associated proteins and carbohydrates. As used herein, the cellular membrane refers to a membrane obtained from a naturally occurring biological membrane of a cell or cellular organelles, or one derived therefrom. As used herein, the term “naturally occurring” refers to one existing in nature. As used herein, the term “derived therefrom” refers to any subsequent modification of the natural membrane, such as isolating the cellular membrane, creating portions or fragments of the membrane, removing and/or adding certain components, such as lipid, protein or carbohydrates, from or into the membrane taken from a cell or a cellular organelle. A membrane can be derived from a naturally occurring membrane by any suitable methods. For example, a membrane can be prepared or isolated from a cell or a virus and the prepared or isolated membrane can be combined with other substances or materials to form a derived membrane. In another example, a cell can be recombinantly engineered to produce “non-natural” substances that are incorporated into its membrane in vivo, and the cellular membrane can be prepared or isolated from the cell to form a derived membrane.

In various embodiments, the cellular membrane covering either of the unilamellar or multilamellar nanoparticles can be further modified to be saturated or unsaturated with other lipid components, such as cholesterol, free fatty acids, and phospholipids, also can include endogenous or added proteins and carbohydrates, such as cellular surface antigen. In such cases, an excess amount of the other lipid components can be added to the membrane wall which will shed until the concentration in the membrane wall reaches equilibrium, which can be dependent upon the nanoparticle environment. Membranes may also comprise other agents that may or may not increase an activity of the nanoparticle. In other examples, functional groups such as antibodies and aptamers can be added to the outer surface of the membrane to enhance site targeting, such as to cell surface epitopes found in cancer cells. The membrane of the nanoparticles can also comprise particles that can be biodegradable, cationic nanoparticles including, but not limited to, gold, silver, and synthetic nanoparticles.

Synthetic or artificial membrane: As used herein, the term “synthetic membrane” or “artificial membrane” refers to a man-made membrane that is produced from organic material, such as polymers and liquids, as well as inorganic materials. A wide variety of synthetic membranes are well known in the art. Cellular membranes as disclosed herein can be a hybrid membrane comprising of two or more different types of cellular membranes or comprising one or more naturally occurring cellular membranes with a synthetic lipid membrane.

Viral membrane: As used herein, the term “membrane derived from a virus” refers to viral envelopes that cover the nucleic acid or protein capsids of a virus, and typically contain cellular membrane proteins derived from portions of the host cell membrane (phospholipid and proteins) and include some viral glycoproteins. The viral envelop fuses with the host's membrane, allowing the capsid and viral genome to enter and infect the host.

Nanoparticle: The term “nanoparticle” as used herein refers to nanostructure, particles, vesicles, or fragments thereof having at least one dimension (e.g., height, length, width, or diameter) of between about 1 nm and about 10 μm. For systemic use, an average diameter of about 50 nm to about 500 nm, or 100 nm to 250 nm may be preferred. The terms “nanostructure” includes, but is not necessarily limited to, particles and engineered features. The particles and engineered features can have, for example, a regular or irregular shape. Such particles are also referred to as nanoparticles. The nanoparticles can be composed of organic materials or other materials, and can alternatively be implemented with porous particles. The layer of nanoparticles can be implemented with nanoparticles in a monolayer or with a layer having agglomerations of nanoparticles. As used herein, the nanoparticle comprises an inner core covered by an outer surface comprising the membrane as discussed herein. The invention contemplates any nanoparticles now known and later developed that can be coated with the membrane described herein.

In certain embodiments, the cell includes, but is not limited to, a blood cell such as a red blood cell (RBC), a white blood cell (WBC), and a platelet, an immune cell, such as a macrophage, a monocyte, a B-cell, and a T-cell, a tumor or cancer cell, and other cells, such as an epithelial cell, an endothelial cell, and a neural cell. In other embodiments, the membrane of the outer surface is derived from non-terminally differentiated or pluripotent stem cells, such as a hematopoietic stem cell, a bone marrow stem cell, a mesenchymal stem cell, a cardiac stem cell, or a neural stem cell. In yet other embodiments, the cellular membrane is derived from a cell component including, but not limited to, an exosome, a secretory vesicle or a synaptic vesicle. In certain embodiments, the outer surface of the nanoparticle of the present invention further comprises a synthetic membrane or synthetic components, along with the naturally derived membrane.

The membranes according to the invention can be obtained and assembled by methods described herein and known in the art, for example, See Desilets et al., Anticancer Res. 21: 1741-47; Lund et al., J Proteome Res 2009, 8 (6), 3078-3090; Graham, Methods Mol Biol 1993, 19, 97-108; Vayro et al., Biochem J 1991, 279 (Pt 3), 843-848; Navas et al., Cancer Res 1989, 49 (8), 2147-2156; Henon et al., C R Acad Sci Hebd Seances Acad Sci D 1977, 285 (1), 121-122; and Boone et al., J Cell Biol 1969, 41 (2), 378-392), the entire contents of which are incorporated by reference herewith.

The present invention provides that the inner core comprises an oxygen delivery vehicle. An oxygen delivery vehicle includes a fluorocarbon compound, which is an organofluorine compound with the formula CxFy, containing at least carbon and fluorine. Compounds with the prefix perfluoro- are hydrocarbons, including those with heteroatoms, wherein the C—H bonds have been replaced by C—F bonds. Fluorocarbons of the present invention include perfluoroalkanes, fluoroalkenes and fluoroalkynes or perfluoroaromatic compounds. In embodiments, the fluorocarbon is a perfluoro halide selected from fluoride, chloride, bromide, iodide and astatide. In embodiments, the perfluoro halide is perfluorooctyl bromide (C8F17Br, also referred to as perflubron), perfluorodecyl bromide (C10F21Br) or perfluorodichlorooctane (C8F16C12).

As used herein, and unless otherwise specified, a compound described herein, such as perfluorocarbon (PFC), is intended to encompass all possible derivatives and stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

The present invention further provides that the invention nanoparticle can comprise a releasable cargo that can be located in any place inside or on the surface of the nanoparticle. In certain embodiments, the releaseable cargo is located within or on the inner core of the inventive nanoparticle. In other embodiments, the releasable cargo is located between the inner core and the outer surface of the inventive nanoparticle. In yet other embodiments, the releasable cargo is located within or on the outer surface of the inventive nanoparticle. A trigger for releasing the releasable cargo from the inventive nanoparticle includes, but is not limited to, a contact between the nanoparticle and a target cell, tissue, organ or subject, or a change of an environmental parameter, such as the pH, ionic condition, temperature, pressure, and other physical or chemical changes, surrounding the nanoparticle.

In certain embodiments, the releasable cargo comprises one or more of a therapeutic agent, prophylactic agent, diagnostic or marker agent, prognostic agent, or a combination thereof. Examples of therapeutic agents include, but are not limited to, an antibiotic, an antimicrobial, a growth factor, a chemotherapeutic agent, or a combination thereof. Exemplary diagnostic or prognostic agents can be an imaging marker. In yet certain other embodiments, the releasable cargo is a metallic particle comprising a gold particle, a silver particle, or an iron oxide particle. In other embodiments, the releasable cargo is a PFC particle. In other embodiments, the releasable cargo is a dendrimer particle or an inorganic particle comprising a silica particle, a porous silica particle, a phosphate calcium particle or a quantum dot, or a metallic particle comprising a gold particle, a silver particle, or an iron oxide particle.

The present invention further provides that the inventive nanoparticle can be in any suitable shape, including, but not limited to, sphere, square, rectangle, triangle, circular disc, cube-like shape, cube, rectangular parallelepiped (cuboid), cone, cylinder, prism, pyramid, right-angled circular cylinder, or other regular or irregular shape, and has a diameter from about 10 nm to about 10 In certain embodiments, the invention nanoparticle has a diameter from about 50 nm to about 500 nm.

The present invention further provides that the nanoparticle can substantially lack constituents of the cell from which the cellular membrane is derived or constituents of the virus from which the viral membrane is derived. In certain embodiments, the nanoparticle of the present invention substantially lacks cytoplasm, nucleus and/or cellular organelles of the cell from which the cellular membrane is derived. In yet certain embodiments, the nanoparticle of the present invention substantially maintains natural structural integrity or activity of the cellular membrane, the membrane derived from a virus or the constituents of the cellular membrane or viral membrane. The structural integrity of the cellular membrane includes primary, secondary, tertiary or quaternary structure of the cellular membrane, the membrane derived from a virus or the constituents of the cellular membrane or viral membrane, and the activity of the cellular membrane includes, but is not limited to, binding activity, receptor activity, signaling pathway activity, and any other activities a normal naturally occurring cellular membrane, the membrane derived from a virus or the constituents of the cellular membrane or viral membrane, would have. In certain embodiments, the nanoparticle of the present invention is biocompatible and/or biodegradable.

The present invention also provides a pharmaceutical composition comprising a medicament delivery system comprising an effective amount of the nanoparticle of the present invention. In certain embodiments, the pharmaceutical composition of the present invention further comprises one or more additional active ingredients, with or without a medically or pharmaceutically acceptable carrier or excipient, that can be administered along with or in combination with the nanoparticle of the present invention.

The present invention further provides administering to the subject in need one or more other active ingredients, with or without a pharmaceutically acceptable carrier or excipient, along or in combination with the aforementioned immunogenic composition or vaccine. The aforementioned immunogenic composition or the vaccine of the present invention, as well as the other active ingredient, can be administered, alone or in combination, via any suitable administration route, including but not limited to oral, nasal, inhalational, parental, intravenous, intraperitoneal, subcutaneous, intramuscular, intradermal, topical, or rectal. In certain embodiments, the immunogenic composition or the vaccine of the present invention, as well as the other active ingredient, is administered via a medicament delivery system to the subject in need. The type of administration route or the type of other active ingredient used herein is not particularly limited.

### Examples

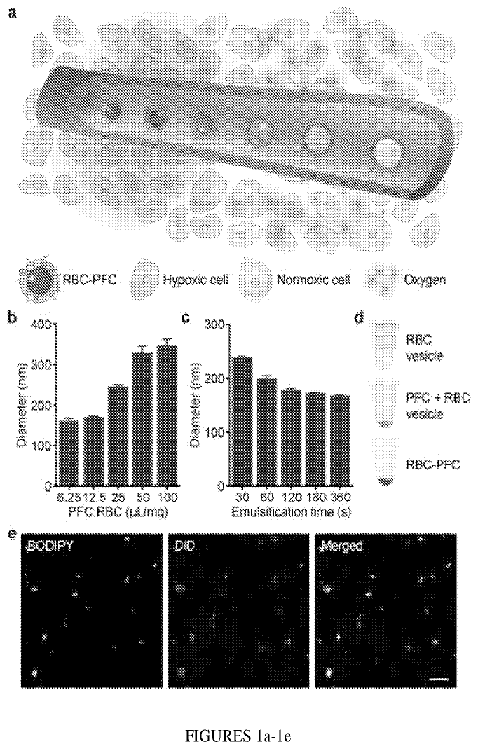

In this example, the invention provides a biomimetic PFC nanoformulation for use as an oxygen delivery vehicle (FIG. 1a). The use of cell membrane coatings is an emerging nanotechnology that has been shown to widely enhance the ability of synthetic nanomaterials to interface with complex biological environments in vivo.^{[19-22]} Cell membrane-coated nanoparticles have been successfully fabricated from a wide range of cell types, and each of them exhibits unique properties that can be leveraged for a variety of applications.^{[23-31]} In particular, the use of RBC coatings has demonstrated exceptional utility for improving biocompatibility and reducing immunogenicity.^{[32-34]} In the present invention, RBC membrane is used to stabilize PFC nanoemulsions (denoted ‘RBC-PFCs’), and the oxygen carrying capacity of the resulting formulation was evaluated. The ability of the RBC-PFCs to reverse hypoxia-induced effects both in vitro and in an animal model of hemorrhagic shock are then demonstrated.

First, it was demonstrated that cell membrane material could be used to facilitate the formation of stable PFC nanoemulsions. For this purpose, the membrane derived from RBCs was chosen, given its previously demonstrated ability to enhance circulation, prevent cellular uptake, and improve immunocompatibility.^{[24, 32, 33, 35]} Perfluorooctyl bromide, which readily forms nanoemulsions,^{[36-38]} was chosen as the model PFC given its widespread study and use as an oxygen carrier.^{[16]} Various amounts of the PFC were mixed with purified RBC membrane and emulsified by sonication (FIG. 1b). It was found that, as the input of the PFC increased, the size of the emulsions also increased, with the final size growing dramatically to above 200 nm at PFC to membrane protein ratios greater than 12.5 μL/mg. At this threshold ratio, it was further shown that the size of the formulation decreased with increasing emulsification times, with the final size reaching below 200 nm after approximately 60 s (FIG. 1c). With further input of energy beyond 2 min, there was a much less pronounced reduction in the final size of the RBC-PFCs. Subsequent studies were conducted using RBC-PFCs fabricated at a PFC to protein ratio of 12.5 μL/mg and with 3 min of sonication.

To confirm that the RBC membrane material was successfully associated with the PFC, the RBC membrane was labeled with a lipophilic far-red fluorescent dye that visually appears blue in color. When the RBC-PFCs were centrifuged at a low speed, significant blue color was observed in the pellet (FIG. 1d, grayscale). In contrast, RBC vesicles alone centrifuged at the same speed did not pellet, leaving all the blue color in the supernatant. Additionally, when PFC was emulsified, followed by mixing with RBC vesicles, significantly less blue color was observed in the pellet, indicating that the spontaneous association between the two components was limited. Overall, this experiment provided a strong indication that, in the final RBC-PFC formulation, the RBC and PFC components were successfully associated together. This was further confirmed by confocal fluorescence microscopy, where a green fluorescent dye was used to label the PFC core in addition to the far-red dye that was used to label the RBC membrane. In this case, significant colocalization of the two fluorescent channels was observed, providing another qualitative indication of a close association between the RBC membrane and PFC components (FIG. 1e). In total, the data indicated that biological membrane could be used to directly facilitate the emulsification of the PFC.

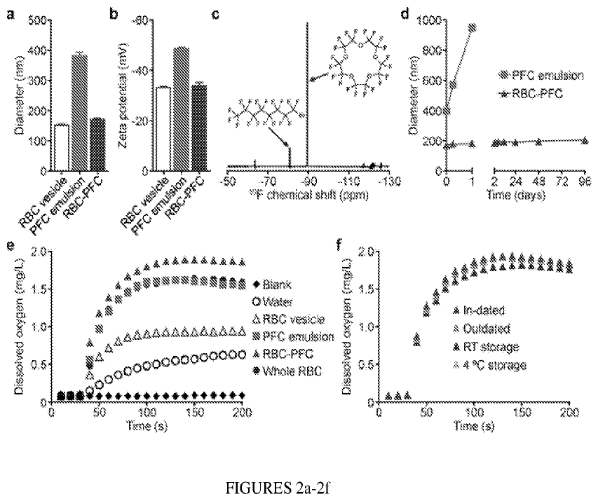

Next, the physicochemical properties of the optimized RBC-PFC formulation were characterized. Dynamic light scattering measurements revealed that the final nanoemulsions were approximately 170 nm in diameter (FIG. 2a). This was significantly smaller than PFC emulsified alone in the absence of RBC membrane, which measured almost 400 nm after synthesis, and it was slightly larger than sonicated RBC membrane, which produced vesicles approximately 150 nm in diameter. The sizing data highlights to role of the RBC membrane as a stabilizer of the PFC during the emulsification process, enabling the formation of smaller sized droplets. Additionally, the surface zeta potential of RBC-PFCs was shown to be near identical to that of RBC vesicles alone, suggesting that the membrane had masked the highly negative zeta potential of the PFC cores (FIG. 2b). These results were consistent with previous works where cell membrane was used to coat negatively charged nanoparticulate cores.^{[25, 39]} Fluorine-19 nuclear magnetic resonance (¹⁹F NMR) was then used to determine the amount of PFC that was retained in the final nanoformulation. RBC-PFCs were spiked with a known concentration of perfluoro-15-crown-5-ether and subjected to ¹⁹F NMR spectroscopy (FIG. 2c). By comparing the integrated area for each characteristic peak and taking into consideration the number of fluorine groups contributing to each group, it was calculated that approximately 62% of the inputted PFC was retained after fabrication of the RBC-PFCs.

In order to test if the RBC-PFC formulation was suitable for long-term storage, its stability in solution was assessed over time (FIG. 2d). The nanoemulsions exhibited little increase in size over the course of 96 days, staying at around 200 nm for the entire duration. In contrast, the non-stabilized PFC emulsions quickly grew after synthesis, reaching nearly 1 μm within 1 day. This data further confirmed that the RBC membrane could serve as a good stabilizer for the PFC and suggested that the two components remained strongly associated with each other over time. To evaluate oxygen delivery capacity, 20 mL of water was first deoxygenated by nitrogen purging. Subsequently, various samples were injected into the closed system and the dissolved oxygen (DO) levels were monitored over time (FIG. 2e). The RBC-PFCs exhibited an exceptional ability to introduce oxygen into the deoxygenated water, elevating DO levels to near 2.0 mg/L, whereas the addition of oxygenated water alone resulted in a level of approximately 0.6 mg/L. The formulation also outperformed PFC emulsions without membrane stabilization; the extra oxygen carrying capacity of the RBC-PFC formulation could likely be attributed to residual membrane-associated hemoglobin present on the RBC vesicles, which alone were slightly better than oxygenated water. Notably, the RBC-PFC formulation also outperformed whole RBCs when normalized to the same amount of membrane. To evaluate the translational potential of the platform, RBC-PFCs were fabricated using both in-dated and just-expired human RBCs obtained from a local blood bank (FIG. 2f). It was found that the dissolved oxygen kinetics between the two formulations were identical. Further, the RBC-PFC nanoemulsions were evaluated after 1 week of storage at either 4° C. or room temperature, and the performance of both samples was nearly identical to freshly made RBC-PFC.

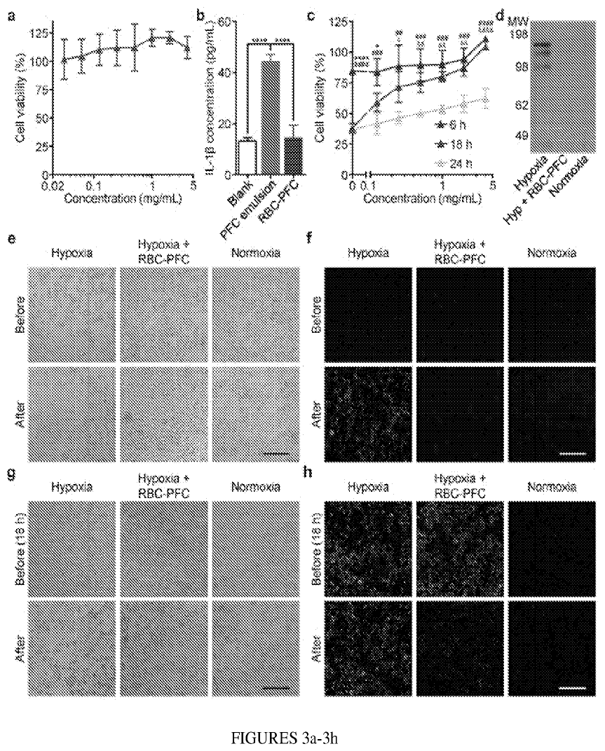

After characterization of the RBC-PFC formulation, the ability of the nanoemulsions to mitigate the effects of hypoxia on cells in vitro was evaluated. First, the safety of the formulation was evaluated on Neuro2a, a murine neuroblastoma cell line, which has previously been used to study the effects of hypoxia.^{[40]} Across the concentrations tested, the RBC-PFC formulation had no harmful impact on cell viability (FIG. 3a). As PFCs are known to exert immunological effects on macrophages,^{[41}] the impact of RBC-PFC on murine J774 macrophages was evaluated (FIG. 3b). Whereas culture with non-stabilized PFC emulsions significantly elevated the level of interleukin 1 beta (IL-1β), an indicator of macrophage activation,^{[42]} culture with RBC-PFC resulted in cytokine levels consistent with baseline. Next, the ability of the RBC-PFC formulation to rescue cells from hypoxic conditions was evaluated. Neuro2a cells were cultured under hypoxia for varying amounts of time, followed by addition of RBC-PFCs. The cells were then cultured for another 24 h, again under hypoxic conditions, and the effects on cell viability were assessed (FIG. 3c). With 6 h of hypoxia induction, the nanoformulation was highly effective in preserving cell viability, with near full efficacy across the concentrations tested. Even after 18 h of induction, full preservation of viability could be achieved when employing RBC-PFCs at high concentrations. After 24 h of hypoxia induction, full viability could no longer be reached, even at the highest amount of nanoemulsions tested, although it was observed that viability trended upwards with increasing concentration.

To further study the impact of RBC-PFCs on cells in vitro, the expression of hypoxia-inducible factor 1-alpha (HIF1α), a characteristic intracellular marker of hypoxia,^{[43]} was evaluated by western blotting analysis (FIG. 3d). When employing 18 h of hypoxia induction, expression of HIF1α could easily be detected in untreated cells, whereas addition of the RBC-PFCs completely abrogated expression of the protein. The western blotting results of cells treated with the nanoformulation were consistent with those from cells cultured under normoxic conditions. The impact of the RBC-PFC formulation could also be readily visualized under brightfield microscopy, with the treated cells looking healthier and denser when compared with untreated cells (FIG. 3e). This was also seen when staining cells with a commercial detection reagent, which could be used to fluorescently visualize hypoxic cells (FIG. 3f). Significant fluorescent signal was observed in cells after being subjected to hypoxia, whereas treatment with RBC-PFCs resulted in the absence of signal. These effects were even more pronounced following an 18 h hypoxia induction period, where the nanoemulsions were able to reverse the effects of exposure to low oxygen levels (FIG. 3g,3h).

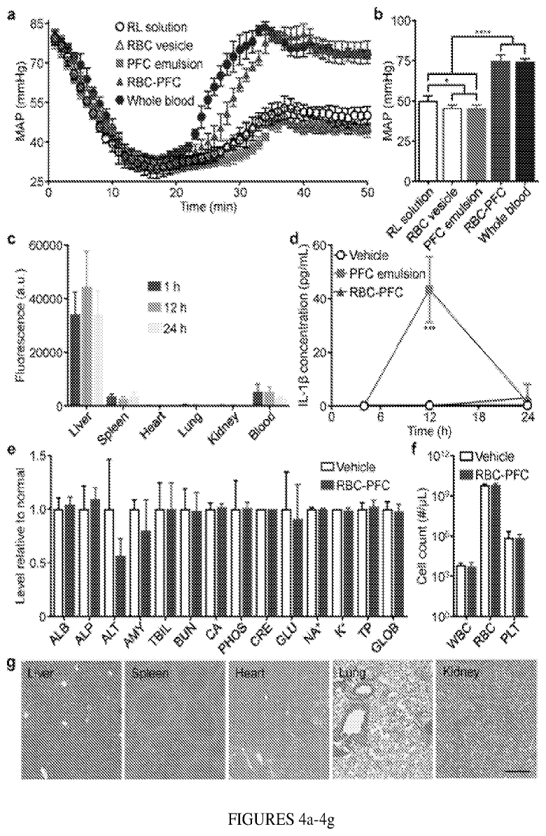

Upon successfully confirming the activity of the RBC-PFC formulation in vitro, an animal model of hemorrhagic shock was used to evaluate in vivo oxygen delivery efficacy.^{[44]} To establish this model, mice were anesthetized, and a femoral artery was cannulated. Blood was then slowly withdrawn from the cannulated artery using a syringe pump such that the mean arterial pressure (MAP) reached a critical level of 35 mmHg After allowing the mice to stabilize for a period of 10 min, various resuscitation fluids were administered via syringe pump, and MAP was monitored over time (FIG. 4a,4b). When reinfused with the as withdrawn whole blood, the MAP value quickly recovered back to baseline levels, which was the expected outcome. This was also the case for the RBC-PFC formulation; the kinetics were slightly delayed compared with whole blood, but the MAP settled at the same final value of approximately 75 mmHg. In contrast, both the PFC emulsion and RBC vesicle controls performed similarly to Ringer's lactate solution, which served as a negative control. For these groups, the MAP values stabilized at just over 45 mmHg, which was still near the critical induction value. The enhanced resuscitation ability of the RBC-PFC formulation may result from its increased oxygen carrying capacity, as well as from the improved stability characteristics bestowed by the cell membrane,^{[24]} which should enhance RBC-PFC blood residence compared with the non-stabilized PFC emulsions.

Finally, we sought to evaluate the in vivo biodistribution of the RBC-PFCs and assess their safety. To study the organ level distribution, the nanoemulsions were labeled with a fluorescent dye, followed by intravenous administration through the tail vein. At set time points, the mice were then euthanized, and the major organs were analyzed for their fluorescent signal (FIG. 4c). From the results, it could be seen that a majority of the RBC-PFCs was found in the liver, with significant amounts also present in the spleen and the blood at all of the time points studied. This pattern of distribution is consistent with other cell membrane-derived formulations.^{[24]} To evaluate safety, a high bolus dose of RBC-PFCs was administered intravenously, followed by tracking of serum IL-1β levels (FIG. 4d). Whereas non-stabilized PFC emulsions elicited a significant spike at 12 h post-administration, cytokine levels after injection of RBC-PFC nanoemulsions remained at baseline levels. A comprehensive analysis of blood chemistry was performed on major blood cell populations 24 h after RBC-PFC administration (FIG. 4e,4f). Compared to mice administered with vehicle only, no statistical difference was observed in any of the parameters that were studied. Subsequently, the major organs were collected and subjected to histological sectioning (FIG. 4g). Analysis after hematoxylin and eosin (H&E) staining revealed normal appearance in all the organs studied, including the liver, spleen, heart, lungs, and kidneys.

In conclusion, the successful fabrication of a natural—synthetic oxygen delivery vehicle consisting of PFC stabilized by cell-derived membrane was demonstrated. The resulting nanoformulation was shown to be highly stable over time and had improved oxygen carrying capacity compared with whole RBCs. Notably, the RBC-PFC formulation was able to attenuate the effects of hypoxia in vitro and was able to fully resuscitate mice in a model of hemorrhagic shock. The platform incorporates the advantages of both component parts, combining the high oxygen carrying capacity of the synthetic PFC with the biocompatibility of the natural cell membrane. It can be envisioned that RBC-PFC fabrication, a facile process that converts RBCs into stable semi-synthetic nanoparticulates, can be employed in the future as a means of prolonging the usefulness of perishable human donations. RBC-PFCs can be synthesized from just-expired RBCs in times of surplus and banked in long-term storage for use during periods of high demand, which would greatly simplify the logistics of blood supply management. As a potent oxygen carrier, these biomimetic nanoemulsions can ultimately help to address an area of significant need in the clinic.

Preparation and Characterization of RBC-PFCs. All animal experiments followed protocols that were reviewed, approved, and performed under the regulatory supervision of the University of California San Diego's Institutional Animal Care and Use Committee (IACUC). Fresh RBCs were purified from whole blood collected from male CD-1 mice (Envigo), and membrane ghosts were obtained by hypotonic lysis.^{[24]} The RBC membrane was suspended at a final protein concentration of 2 mg/mL for further use. To prepare RBC-PFCs, varying volumes of the PFC perfluorooctyl bromide (Sigma Aldrich) were mixed with 2 mL of RBC membrane solution, followed by emulsification on ice using a Fisher Scientific 150E Digital Sonic Dismembrator for increasing amounts of time with an on/off interval of 2 s/1 s. The resulting RBC-PFCs were centrifuged at 600 g for 5 min to remove excess membrane vesicles, followed by resuspension in water or the appropriate media. Size and zeta potential measurements were conducted by dynamic light scattering using a Malvern Instruments Zetasizer Nano ZS. Following the optimization experiments, the final RBC-PFC formulation was fabricated at a ratio of 12.5 μL PFC per 1 mg of RBC membrane protein with 3 min of emulsification. All stated RBC-PFC concentrations are expressed in terms of the protein content of the formulation. RBC vesicle and PFC emulsion controls were fabricated by sonicating the individual components for 3 min. For the stability study, samples were stored at room temperature, and size was measured periodically.

Imaging of RBC-PFCs. To label the RBC membrane with 1,1′-dioctadecyl-3,3,3′,3′-tetramethylindodicarbocyanine perchlorate (DiD, excitation/emission=644/665 nm; Invitrogen), the dye was added to the membrane solution at a final concentration of 10 μg/mL. For dye staining of the PFC core, BODIPY FL iodoacetamide (excitation/emission=503/512 nm; Invitrogen) was modified with 3,3,4,4,5,5,6,6,7,7,8,8,9,9,10,10,10-heptadecafluoro-1-decanethiol (Sigma Aldrich) using a previously reported approach.^{[45]} The resulting conjugate was then dissolved at 0.2 mg/mL in the PFC. For fluorescent imaging of the RBC-PFCs, dual-labelled samples were immobilized on glass slides with Tissue-Tek OCT compound (Sakura Finetek) and visualized using an Olympus FV1000 confocal microscope.

PFC Loading Quantification. In order to quantify the loading of PFC into the final RBC-PFC formulation, Triton X-100 (Sigma Aldrich) was added at a final ratio of 0.5% to disrupt the RBC membrane. The PFC was then extracted by mixing the lysed RBC-PFC solution with an equal volume of deuterated chloroform (Sigma Aldrich). As an internal standard, 2 μL of perfluoro-15-crown-5-ether (Sigma Aldrich) was added to 1 mL of the chloroform fraction. The sample was then subject to ¹⁹F-NMR on a JEOL ECA 500 NMR spectrometer. Data analysis was performed using Mestrelab Research MestReNova software.

Dissolved Oxygen Kinetics. A measurement apparatus was built by covering a 100 mL beaker with a foam cap, which was sealed in place using Parafilm M (Bemis). Three holes were cut into the foam cap in order to accommodate a temperature probe, an oxygen probe, and a glass pipette for nitrogen purging. Before the start of each experiment, 20 mL of water was added into the beaker, equilibrated to 37° C., and purged with nitrogen to remove dissolved oxygen. Then, 2 mL of RBC-PFCs at 2 mg/mL was injected into the system 30 s after nitrogen flow shutdown, and the dissolved oxygen values were monitored using a Hanna Instruments edge dedicated dissolved oxygen meter. RBC vesicle and PFC emulsion controls were employed at concentrations equivalent to the RBC-PFC formulation. The whole RBC sample was used at an RBC content with equivalent membrane protein compared with the RBC-PFC samples. In-dated and outdated (2 days post-expiration) human O-positive RBCs were obtained from the San Diego Blood Bank.

In Vitro Toxicity and Hypoxia Studies. Murine neuroblastoma Neuro2a cells (CCL-131; American Type Culture Collection) and J774 macrophages (TIB-67; American Type Culture Collection) were maintained in Dulbecco's modified eagle medium (HyClone) supplemented with 10% fetal bovine serum (HyClone) and 1% penicillin-streptomycin (Gibco). To assess the toxicity of RBC-PFCs, Neuro2a cells were seeded in 96-well plates at 1×10⁴ cells per well. The cells were then incubated under normoxic conditions (20% O₂/5% CO₂/75% N₂) in a Thermo Scientific Heracell 150i incubator with RBC-PFCs at various concentrations. After 24 h, cell viability was quantified using a CellTiter AQᵤₑₒᵤₛ One Solution cell proliferation assay (Promega) following the manufacturer's instructions. To evaluate the potential immunological impact of the nanoformulation, RBC-PFC was incubated with J774 cells at a concentration of 4 mg/mL. A PFC emulsion control was employed at an equivalent concentration. At 24 h, the culture medium was collected, and cytokine concentrations were assessed using a mouse IL-1β ELISA kit (Biolegend) per the manufacturer's instructions.

For the hypoxia treatment study, Neuro2a cells were cultured under hypoxic conditions (1% O₂/5% CO₂/94% N₂) in a Thermo Scientific Forma Series 3 WJ incubator for various induction periods. Afterwards, the media was replaced with fresh media containing various RBC-PFC concentrations and the cells were kept under hypoxic conditions for another 24 h before assessing cell viability. For the imaging studies, Image-iT Green hypoxia reagent (Invitrogen) was added to the cells at a final concentration of 5 μM for 30 min before washing the cells with fresh media. The cells were then subject to various hypoxia induction periods before the media was replaced with fresh media containing RBC-PFCs at 4 mg/mL. Cells were incubated for another 6 h and 24 h under hypoxic conditions before imaging under fluorescence and brightfield microscopy, respectively, using a Thermo Fisher Scientific EVOS FL cell imaging system. The control normoxia group was cultured under normoxic conditions for the duration of the experiment.

To assess the levels of HIF1α, Neuro2a cells were seeded at 5×10⁵ cells per well in 6-well plates. Cells were incubated under hypoxic conditions for 18 h, after which the media was replaced with fresh media, either with or without 4 mg/mL of RBC-PFCs. The cells were then cultured for another 24 h under hypoxic conditions. The normoxia group stayed under normoxic conditions for the duration of the experiment. Afterwards, the cells were lysed on ice with RIPA buffer (Sigma Aldrich), supplemented with 1% 0.5 M ethylenediaminetetraacetic acid (Invitrogen) and 1% of a protease inhibitor cocktail (Sigma Aldrich). Lysed cells were then scraped off the wells and centrifuged at 14,000 g, after which the supernatant was collected. Protein concentrations were normalized to 1 mg/mL. The samples were prepared using NuPAGE 4× lithium dodecyl sulfate sample loading buffer (Invitrogen) and then run on 12-well Bolt 4-12% bis-tris minigels (Invitrogen) in MOPS running buffer (Invitrogen). After transferring to 0.45 μm nitrocellulose membrane (Pierce) in Bolt transfer buffer (Invitrogen) at 10 V for 60 min, the membranes were blocked with 5% bovine serum albumin (Sigma Aldrich) in phosphate buffered saline (PBS, Mediatech) with 0.05% Tween 20 (National Scientific). The blots were then incubated with anti-HIF1α (28b; Santa Cruz Biotechnology), followed by the appropriate horse radish peroxidase-conjugated secondary antibody (Biolegend). ECL western blotting substrate (Pierce) and a Mini-Medical/90 developer (ImageWorks) were used to develop and image the blots.

In Vivo Hemorrhagic Shock Treatment. To evaluate efficacy in a hemorrhagic shock model, 6-week-old male CD-1 mice were intraperitoneally administered with a cocktail of ketamine (Pfizer) at 100 mg/kg and xylazine (Lloyd Laboratories) at 20 mg/kg. Anesthesia was maintained for the entire surgical procedure and the mice were kept on a 37° C. heat pad. A 0.5 cm incision parallel to where the right femoral artery runs in the groin area between the abdomen and thigh was made using a small surgical scissor. The femoral artery was carefully isolated, and a small incision was then cut into the artery so that PE-10 tubing (Braintree Scientific) primed with 0.3% heparin (Sigma Aldrich) in PBS could be inserted as the cannula. The tubing was connected to a Digi-Med BPA-400 blood pressure analyzer for the continuous monitoring of MAP. The left femoral artery was cannulated in a similar fashion and connected to a Kent Scientific GenieTouch syringe pump to perform the hemorrhagic shock and resuscitation procedure. To induce hemorrhage, blood was steadily withdrawn from the left femoral artery at a constant rate of 0.1 mL/min until the MAP reached 35 mmHg, after which the mice were allowed to stabilize for 10 min. For resuscitation, 1 mL of RBC-PFCs at 2 mg/mL was infused into the left femoral artery at a constant speed of 0.1 ml/min. Ringer's lactate (Fisher Scientific) was used as a negative control, and the withdrawn whole blood was reinfused as a positive control. RBC vesicles and PFC emulsions were used at concentrations equivalent to the RBC-PFC formulation. Solutions that were not isotonic were adjusted to the appropriate osmolarity using concentrated sucrose (Sigma Aldrich). The mice were monitored for another 20 min after completion of the infusion. MAP values were recorded for the duration of the study, and the mice were euthanized immediately afterwards.

RBC-PFC In Vivo Biodistribution and Safety. To study the biodistribution, DiD-labeled RBC-PFCs with 1.6 mg of protein content was administered via the tail vein. At 1, 4, and 24 h, mice were euthanized and their major organs, including the liver, spleen, heart, lungs, kidneys, and blood were collected and weighed. The organs were then homogenized in 1 mL of PBS using a Biospec Mini-Beadbeater-16. Fluorescence was read using a Tecan Infinite M200 plate reader. Total weight of blood was estimated as 6% of mouse body weight. To assess serum cytokine levels, RBC-PFCs with 4 mg of protein content, or an equivalent amount of PFC emulsions, were intravenously administered. At 4, 12, and 24 h after administration, blood was sampled by submandibular puncture, and cytokine levels were assessed using a mouse IL-1β ELISA kit per the manufacturer's instructions. For the other safety studies, RBC-PFCs with 4 mg of protein content was administered intravenously, and after 24 h the blood and major organs were collected for analysis. For the comprehensive metabolic panel, aliquots of blood were allowed to coagulate, and the serum was collected by centrifugation. For the blood counts, blood was collected into potassium-EDTA collection tubes (Sarstedt). Lab tests were performed by the UC San Diego Animal Care Program Diagnostic Services Laboratory. For histological analysis, the major organs were sectioned and stained with hematoxylin and eosin (Leica Biosystems), followed by imaging using a Hamamatsu Nanozoomer 2.0-HT slide scanning system.

### REFERENCES

[1] P. L. Giangrande, Br. J. Haematol. 2000, 110, 758.
[2] L. T. Goodnough, M. E. Brecher, M. H. Kanter, J. P. AuBuchon, N. Engl. J. Med. 1999, 340, 438.
[3] L. M. Napolitano, S. Kurek, F. A. Luchette, H. L. Corwin, P. S. Barie, S. A. Tisherman, P. C. Hebert, G. L. Anderson, M. R. Bard, W. Bromberg, W. C. Chiu, M. D. Cipolle, K. D. Clancy, L. Diebel, W. S. Hoff, K. M. Hughes, I. Munshi, D. Nayduch, R. Sandhu, J. A. Yelon, Crit. Care. Med. 2009, 37, 3124.
[4] L. T. Goodnough, A. Shander, Anesthesiology 2012, 116, 1367.
[5] A. Tinmouth, L. Macdougall, D. Fergusson, M. Amin, I. D. Graham, P. C. Hebert, K. Wilson, Arch. Intern. Med. 2005, 165, 845.
[6] J. Belien, H. Force, Eur. J. Oper. Res. 2012, 217, 1.
[7] P. W. Buehler, F. D'Agnillo, D. J. Schaer, Trends Mol. Med. 2010, 16, 447.
[8] J. Y. Chen, M. Scerbo, G. Kramer, Clinics (Sao Paulo) 2009, 64, 803.
[9] C. I. Castro, J. C. Briceno, Artif. Organs 2010, 34, 622.
[10] T. Henkel-Honke, M. Oleck, AANA J. 2007, 75, 205.
[11] R. K. Spence, E. D. Norcross, J. Costabile, S. Mccoy, A. C. Cernaianu, J. B. Alexander, M. J. Pello, U. Atabek, R. C. Camishion, Artif. Cells Blood Substit. Biotechnol. 1994, 22, 955.
[12] J. G. Riess, Artif. Cells Blood Substit. Biotechnol. 2006, 34, 567.
[13] K. C. Lowe, Comp. Biochem. Phys. A 1987, 87, 825.
[14] K. C. Lowe, Tissue Eng. 2003, 9, 389.
[15] J. G. Riess, Artif. Cells Blood Substit. Biotechnol. 2005, 33, 47.
[16] K. C. Lowe, J. Mater. Chem. 2006, 16, 4189.
[17] P. M. Winter, K. Cai, S. D. Caruthers, S. A. Wickline, G. M. Lanza, Expert Rev. Med. Devices 2007, 4, 137.
[18] Y. Cheng, H. Cheng, C. Jiang, X. Qiu, K. Wang, W. Huan, A. Yuan, J. Wu, Y. Hu, Nat. Commun. 2015, 6, 8785.
[19] R. H. Fang, Y. Jiang, J. C. Fang, L. Zhang, Biomaterials 2017, 128, 69.
[20] R. H. Fang, A. V. Kroll, W. Gao, L. Zhang, Adv. Mater. 2018, 30, 1706759.
[21] A. V. Kroll, R. H. Fang, L. Zhang, Bioconjug. Chem. 2017, 28, 23.
[22] D. Dehaini, X. Wei, R. H. Fang, S. Masson, P. Angsantikul, B. T. Luk, Y. Zhang, M. Ying, Y. Jiang, A. V. Kroll, W. Gao, L. Zhang, Adv. Mater. 2017, 29, 1606209.
[23] C. M. Hu, R. H. Fang, K. C. Wang, B. T. Luk, S. Thamphiwatana, D. Dehaini, P. Nguyen, P. Angsantikul, C. H. Wen, A. V. Kroll, C. Carpenter, M. Ramesh, V. Qu, S. H. Patel, J. Zhu, W. Shi, F. M. Hofman, T. C. Chen, W. Gao, K. Zhang, S. Chien, L. Zhang, Nature 2015, 526, 118.
[24] C. M. Hu, L. Zhang, S. Aryal, C. Cheung, R. H. Fang, L. Zhang, Proc. Natl. Acad. Sci. USA 2011, 108, 10980.
[25] R. H. Fang, C. M. Hu, B. T. Luk, W. Gao, J. A. Copp, Y. Tai, D. E. O'Connor, L. Zhang, Nano Lett. 2014, 14, 2181.
[26] S. Thamphiwatana, P. Angsantikul, T. Escajadillo, Q. Zhang, J. Olson, B. T. Luk, S. Zhang, R. H. Fang, W. Gao, V. Nizet, L. Zhang, Proc. Natl. Acad. Sci. USA 2017, 114, 11488.
[27] C. M. Hu, R. H. Fang, J. Copp, B. T. Luk, L. Zhang, Nat. Nanotechnol. 2013, 8, 336.
[28] C. M. Hu, R. H. Fang, B. T. Luk, L. Zhang, Nat. Nanotechnol. 2013, 8, 933.
[29] M. Gao, C. Liang, X. Song, Q. Chen, Q. Jin, C. Wang, Z. Liu, Adv. Mater. 2017, 29, 1701429.
[30] X. Wei, J. Gao, F. Wang, M. Ying, P. Angsantikul, A. V. Kroll, J. Zhou, W. Gao, W. Lu, R. H. Fang, L. Zhang, Adv. Mater. 2017, 29, 1701644.
[31] A. V. Kroll, R. H. Fang, Y. Jiang, J. Zhou, X. Wei, C. L. Yu, J. Gao, B. T. Luk, D. Dehaini, W. Gao, L. Zhang, Adv. Mater. 2017, 29, 1703969.
[32] B. T. Luk, R. H. Fang, C. M. J. Hu, J. A. Copp, S. Thamphiwatana, D. Dehaini, W. W. Gao, K. Zhang, S. L. Li, L. F. Zhang, Theranostics 2016, 6, 1004.
[33] L. Rao, L. L. Bu, J. H. Xu, B. Cai, G. T. Yu, X. L. Yu, Z. B. He, Q. Q. Huang, A. Li, S. S. Guo, W. F. Zhang, W. Liu, Z. J. Sun, H. Wang, T. H. Wang, X. Z. Zhao, Small 2015, 11, 6225.
[34] W. Gao, C. M. Hu, R. H. Fang, B. T. Luk, J. Su, L. Zhang, Adv. Mater. 2013, 25, 3549.
[35] C. M. J. Hu, R. H. Fang, B. T. Luk, K. N. H. Chen, C. Carpenter, W. W. Gao, K. Zhang, L. F. Zhang, Nanoscale 2013, 5, 2664.
[36] T. K. Stevens, R. M Ramirez, A. Pines, J. Am. Chem. Soc. 2013, 135, 9576.
[37] Y. T. Lim, Y. W. Noh, J. N. Kwon, B. H. Chung, Chem. Commun. (Camb) 2009, 6952.
[38] C. Giraudeau, B. Djemai, M. A. Ghaly, F. Boumezbeur, S. Meriaux, P. Robert, M. Port, C. Robic, D. Le Bihan, F. Lethimonnier, J. Valette, NMR Biomed. 2012, 25, 654.
[39] J. A. Copp, R. H. Fang, B. T. Luk, C. M. J. Hu, W. W. Gao, K. Zhang, L. F. Zhang, Proc. Natl. Acad. Sci. USA 2014, 111, 13481.
[40] X. W. Liu, Y. Zi, Y. E. Liu, Y. B. Zhang, L. B. Xiang, M. X. Hou, Neurosci. Lett. 2015, 595, 74.
[41] C. S. Cohn, M. M. Cushing, Crit. Care Clin. 2009, 25, 399.
[42] G. Lopez-Castejon, D. Brough, Cytokine Growth Factor Rev. 2011, 22, 189.
[43] J. E. Ziello, I. S. Jovin, Y. Huang, Yale J. Biol. Med. 2007, 80, 51.
[44] Y. Wang, L. L. Wang, W. L. Yu, D. W. Gao, G. X. You, P. L. Li, S. Zhang, J. Zhang, T. Hu, L. Zhao, H. Zhou, Biotechnol. Progr. 2017, 33, 252.
[45] J. Laudien, D. Naglav, C. Grobeta-Heitfeld, K. B. Ferenz, H. de Groot, C. Mayer, S. Schulz, A. Schnepf, M. Kirsch, J. Microencapsul. 2014, 31, 738.

1. A nanoparticle comprising:
a) an inner core comprising an oxygen delivery vehicle comprising a perfluorocarbon (PFC); and
b) an outer surface comprising a cellular membrane or hybrid membrane derived from a cell.

2. The nanoparticle of claim 1, wherein the PFC is perfluorooctyl bromide or other perfluoro halide.

3. The nanoparticle of claim 1, wherein the cellular membrane or hybrid membrane is derived from a blood cell, an immune cell, a stem cell, an endothelial cell, an exosome, a secretory vesicle or a synaptic vesicle.

4. The nanoparticle of claim 3, wherein the cellular membrane or hybrid membrane comprises a plasma membrane derived from a red blood cell.

5. The nanoparticle of claim 1, which further comprises a releasable cargo.

6. The nanoparticle of claim 5, wherein the releasable cargo is a therapeutic agent, a prophylactic agent, a diagnostic or marker agent, a prognostic agent, or a combination thereof.

7. The nanoparticle of claim 3, wherein the cellular membrane or hybrid membrane comprises a membrane derived from a white blood cell.

8. The nanoparticle of claim 7, wherein the cellular membrane or hybrid membrane comprises a membrane derived from a macrophage.

9. The nanoparticle of claim 3, wherein the cellular membrane or hybrid membrane comprises a membrane derived from a platelet.

10. A medicament delivery system, which comprises an effective amount of the nanoparticle of claim 1.

11. A pharmaceutical composition comprising an effective amount of the nanoparticle of claim 1 and a pharmaceutically acceptable carrier or excipient.

12. A method for treating or preventing a disease or condition in a subject in need comprising administering to said subject an effective amount of the nanoparticle of claim 1.

13. The method of claim 12, wherein the disease or condition is decompression sickness, sickle cell crises, surgery, trauma, cancer oxygen sensitizer, or other hypoxia related condition.

14. A process for making a nanoparticle comprising:
a) combining an inner core comprising perfluorocarbon (PFC), and an outer surface comprising a cellular membrane or hybrid membrane derived from a cell; and
b) exerting exogenous energy on the combination to form a nanoparticle comprising said inner core and said outer surface.

15. The process of claim 14, wherein the PFC is perfluorooctyl bromide or other perfluoro halide.

16. The process of claim 14, wherein the cellular membrane or hybrid membrane is derived from a blood cell, an immune cell, a stem cell, an endothelial cell, an exosome, a secretory vesicle or a synaptic vesicle.

17. The nanoparticle of claim 14, wherein the cellular membrane or hybrid membrane comprises a plasma membrane derived from a red blood cell.

18. The nanoparticle of claim 14, wherein the cellular membrane or hybrid membrane comprises a membrane derived from a white blood cell.

19. The nanoparticle of claim 18, wherein the cellular membrane or hybrid membrane comprises a membrane derived from a macrophage.

20. The nanoparticle of claim 14, wherein the cellular membrane or hybrid membrane comprises a membrane derived from a platelet.
